(19) United States
(12) Patent Application Publication
(10) Pub. No.: US 2009/0047298 A1
(43) Pub. Date: Feb. 19, 2009

(54) IMMUNE SYSTEM STIMULANT AND PROCESS OF MANUFACTURING THE SAME

(76) Inventor: David Murray MacDonald, Cumbria (GB)

Correspondence Address: MACMILLAN SOBANSKI & TODD, LLC, ONE MARITIME PLAZA FIFTH FLOOR, 720 WATER STREET, TOLEDO, OH, 43604-1619, US

(21) Appl. No.: 12/279,745

(22) PCT Filed: Feb. 26, 2007
(86) PCT No.: PCT/GB2007/050084

§ 371 Date: Aug. 18, 2008

(30) Foreign Application Priority Data
Feb. 24, 2006 (GB) 0603749.3

(51) Int. Cl. A61K 39/00

(52) U.S. Cl. 424/184.1

(57) ABSTRACT

An immune system stimulant for stimulating the production of antibodies against a pathogen comprises said pathogen attenuated by exposure to hydroxyl radicals. The invention also provides a process for the manufacture of an immune system stimulant.

### SUMMARY OF THE INVENTION

The present invention relates to an immune system stimulant and process for manufacturing the same, and in particular to the creating of a stimulant the action of which mimics natural responses to pathogen attacks.

### BACKGROUND OF THE INVENTION

Vaccination is a technique originally developed by Pasteur where a killed or attenuated strain of a pathogen is administered in order to provoke an immune response that persists for some time. Pasteur observed that if infected by cowpox, the patient had immunity against small-pox and from this has developed the technique of administering a vaccine to promote immunity. The technique of developing a killed or attenuated strain of a pathogen and administering it usually by subcutaneous injections is complex in that the development and testing of the specific vaccine is a lengthy process and the potential side-effects can be significant. Since viruses, in particular, mutate rapidly and animal viruses can make transgenic transfer, the time taken to develop an effective vaccine mitigates against the use of vaccination except against stable diseases.

Vaccination exposes a being (human or animal) to a pathogen, be that bacterial, fungal or viral in an amount that is sufficient to cause the being's antibodies to attack the pathogen but at a level insufficient to overwhelm antibodies mobilized against the attack. In some circumstances, the pathogen is dead, whilst in other circumstances the pathogen is live. There is increasing resistance by individuals to vaccination using live vaccines, particularly where more than one vaccine is administered at the same time.

The present invention has been prompted by the epidemiology of hospital acquired infections, especially methicillin resistant staphylococcus aureus (MRSA). Examination of statistics shows that patients admitted to hospital other than from other institutions show low risk for the first four days of their stay. After four days, risk rises substantially linearly to reach a maximum risk after 15 days. Risk neither rises nor falls after the 15 day period has elapsed.

There is little evidence to support the supposition that compromised immunity has an effect on infection risk: patients with good immunity admitted for orthopaedic operations were at least as much at risk as medical patients.

Whilst MRSA is becoming a major problem in hospitals there is little evidence of Staphylococci aureus or resistant Staphylococci aureus causing anything more than localized infection or inflammation in the general population. There is some evidence of individuals who are bed-ridden or confined in closed spaces being at somewhat higher risk. It would therefore appear that in the general population there is a good deal of immunity to Staphylococci aureus.

Mammals are almost exclusively at risk from pathogens in the atmosphere: pathogens that enter the host through the mouth encounter extremely adverse conditions and are unlikely to survive whilst penetration through the skin can only result from some form of accidental damage.

The invention is based on the hypothesis that a form of limited environmental immunity having a defined decay cycle exists and that this limited environmental immunity is triggered by pathogens in the atmosphere. Further the invention is based on the hypothesis that the immunity is due to the creation of antibodies having a short decay life, and that if the population remains below a trigger threshold production of other antibodies and as a result the creation of memory cells does not occur. If the threat posed by the pathogens in the atmosphere does not materialize the short decay time of the IgM antibody results in the specific immunity lapsing. In this way mammals are adapted to cope with pathogen threats as they arise.

It follows that in a hospital environment it is the above-mentioned limited environmental immunity which defends the being against infection during the first four day period, but thereafter the IgM antibody cannot defend against continued exposure to the levels of MRSA pathogen present in the hospital environment. It follows that the mechanism by which localized or environmental immunity (the creation of specific IgM antibodies) is related to the biocidal effects of open air which are not present in enclosed spaces.

Atmospheric ozone reacts with oleofins in the atmosphere to produce hydroxyl radicals which are highly reactive and kill pathogens in the atmosphere. The process relies on what has been termed the “Open Air Factor”, which is now known to be the hydroxyl radical. This process is described in detail in United Kingdom patent no 1278043. Of course, in enclosed spaces there is no open air factor, although this can be re-created using the method and apparatus described in International Patent Publication No WO 2005/026044. It is also known that cells such as macrophages present in, for example, the lung mucosa, emit ozone when stimulated and work done primarily by a researcher at the Scripps institute at La Jolla in Calif. have shown that the mechanism by which macrophages kill invasive pathogens is the conversion of ozone releasing the hydroxyl radical.

It would therefore be desirable to provide an immune system stimulant.

It would also be desirable to provide a process for manufacture of an immune system stimulant to produce an immune response to a specific pathogen threat.

### SUMMARY OF THE INVENTION

Surprisingly, it has been found that upon exposure of an individual to a pathogen which has been attenuated by exposure to hydroxyl radicals the IgM response is substantially the same as the IgM response upon exposure of an individual to the pathogen which has not been attenuated.

Exposure to sufficient a level of the immune system stimulant of the invention results in the production of IgG antibodies.

According to the present invention there is provided a process for the manufacture of an immune system stimulant as specified in Claim 1.

According to another aspect of the invention there is provided an immune system stimulant as specified in Claim 7.

The invention also provides for the use of a pathogen attenuated by exposure to hydroxyl radicals as specified in Claim 9.

The invention further provides for the use of a pathogen attenuated by exposure to hydroxyl radicals as specified in Claim 10.

The invention further provides for the stimulation of the immune system of an individual as specified in Claim 11.

### BRIEF DESCRIPTION OF THE DRAWINGS

In the drawings, which illustrate preferred features of the invention, and are by way of example:

FIG. 1 is a schematic illustration of a chamber for administration of an immune stimulant of the invention;

FIG. 2 is a schematic illustration of an apparatus for the production of an immune stimulant according to the invention; and

FIG. 3 is a schematic illustration of an inhaler for administration of an immune stimulant to humans.

### DETAILED DESCRIPTION OF THE PREFERRED EMBODIMENTS

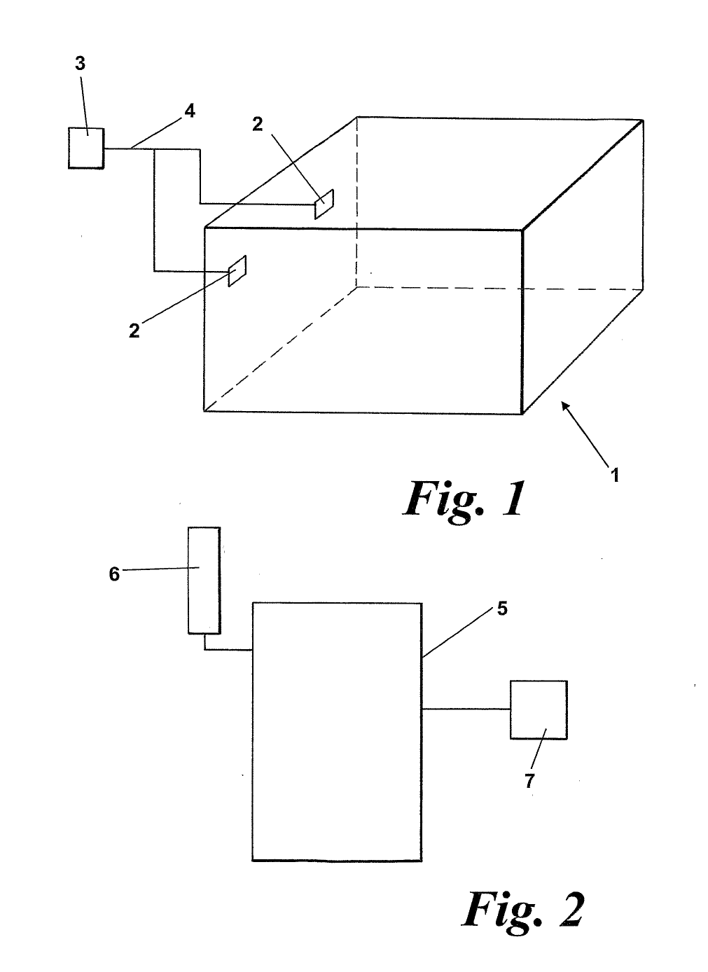

Referring now to FIG. 1, there is shown an enclosed space 1 the atmosphere of which can be accurately controlled. Individuals (in the case of the examples which follow, pigs) are housed in the space 1. Whilst housed in the space 1 the pigs breathe the gaseous mixture of air supply 3 which is connected to air vents 2 via conduits 4. The gaseous mixture is manufactured according to the invention and includes pathogens attenuated by exposure to hydroxyl radicals, which exposure gives rise to an immune system response of the pigs in the form of production of IgM antibodies which act to counter a threat of the same pathogen in its unattenuated form.

FIG. 2 illustrates the apparatus for manufacturing the killed pathogens, which comprises a glass lined chamber 5 which is filled with sterile air carrying a known pathogen previously cultured in a laboratory for example. The target pathogen is introduced into the chamber by a nebuliser 6. The output of a hydroxyl radical generating machine 7 is attached to the interior of the chamber 5. The pathogens are exposed to hydroxyl radicals in sufficient quantity, and for a period long enough to ensure that the proportion of unattenuated pathogens post exposure to hydroxyl radicals is less than the concentration of the pathogen required to cause infection in an individual. Preferably fewer than 0.0001 per centum of the organisms remain viable.

The hydroxyl radicals are preferably produced by the decay of gaseous triplet oxygen (ozone) with a suitable olefine, which may be myrcene or terpinene.

An all glass impinger is used to sample the contents of the chamber 5 in order that the sample may be tested to ensure a satisfactory attenuation of the pathogens, i.e. the remaining number of live pathogens is below infectious limits.

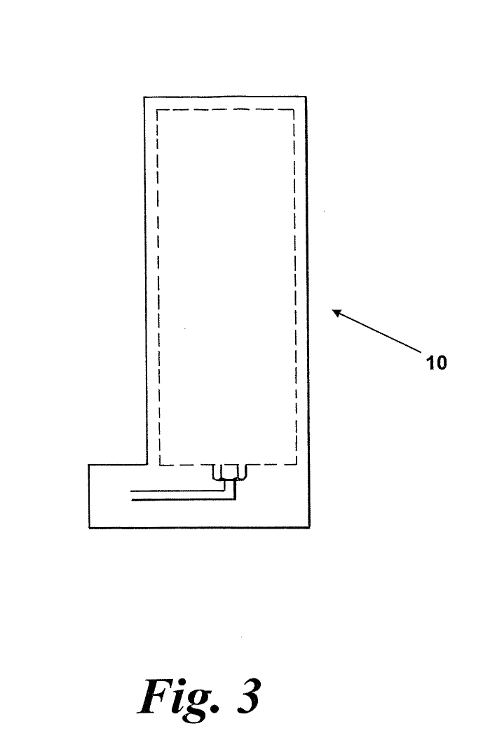

If the sample proves satisfactory the contents of the chamber are collected. For use by humans the said contents are used to fill suitable devises for administration via the respiratory tract, for example an inhaler 10 of the type used by sufferers of asthma, an example of which is illustrated in FIG. 3. Where the said contents are to be administered to animals, alternative devices may be required, one such device being that illustrated in FIG. 1.

One preferred process of the invention comprises the steps of:

1) Identifying and capturing a target pathogen, which may be bacterial, fungal or viral;

2) Culturing the pathogen to produce a significant number of target pathogens;

3) Introducing the cultured pathogen into a chamber;

4) Exposing the cultured pathogens to hydroxyl radicals;

5) Sampling the cultured pathogens following exposure to hydroxyl radicals

6) Capturing the cultured pathogens following exposure to hydroxyl radicals.

### EXAMPLES

Two experiments were performed, both using piglets.

In the first experiment a group of piglets was confined in a chamber having a controlled atmosphere. The atmosphere was treated with a nebulised flow of bacillus subtilis. Blood samples were taken from the piglets post exposure to bacillus subtilis and it was established that a measurable level of IgM production occurred, i.e. exposure to the pathogen resulted in an immune response in the form of production of IgM antibodies.

In a second experiment piglets were divided into four groups. The first group was exposed to bacillus subtilis in the manner as described above with reference to experiment 1. The second group was exposed to bacillus subtilis killed by hydroxyl radicals. The third group was exposed to an atmosphere subjected only to hydroxyl radicals, i.e. not loaded with bacillus subtilis. The fourth group was a control.

Blood samples were taken before the experiment and then daily for three days and thereafter on the seventh and tenth days after commencing exposure. Piglets found to have bacillus subtilis antibodies before exposure were removed from the experiment.

### Results

There was no mean difference between antibody production in the animals exposed to live bacillus subtilis and the animals exposed to an atmosphere where the pathogen had been killed by the hydroxyl radical.

### Conclusions

By exposing an individual to pathogens which have been subjected to hydroxyl radicals the individual's immune response in the form of production of IgM antibodies is mobilized. The production of IgM antibodies can be maintained by continued or repeated exposure of the individual to the attenuated pathogens. Absent the attenuated pathogens, the individual would react by producing IgM antibodies. However, if these are not produced in sufficient number the IgM antibodies are overwhelmed and the individual's next defence mechanism would be required to attack the pathogen with a different antibody type.

Using the process of the invention an immune response stimulant can be manufactured very simply and quickly. Therefore diseases can be treated very quickly. This will be extremely important for the treatment of viruses which mutate.

For both human and animal use, delivery of the stimulant is simple. In the case of humans the stimulant can be administered using a nebuliser, whereas for animals they may be housed within a controlled environment and the atmosphere “fogged” with pathogens attenuated by exposure to hydroxyl radicals.

The process of the invention allows immune system stimulants to be developed against any pathogen extremely quickly. By exposing the isolated pathogen to hydroxyl radicals the pathogens are attenuated such that they do not cause infection when introduced to living beings, yet the immune system of the being responds as if it were being attacked by the pathogen in its unattenuated form producing IgM antibodies. Important conditions such as MRSA, or viral conditions such as HIV/AIDS, influenza, avian influenza, foot and mouth may be countered using the immune system stimulant according to the invention.

In addition to generating an immune system response, the stimulant of the invention may be used to cause vaccination of an individual. Where a threshold level of IgM production is exceeded, for example where the individual is subject to a significant and sustained threat of actual infection, decaying IgM's trigger an IgG response. The IgG antibody has memory protecting the individual against further attack by the same pathogen. Using the stimulant of the invention at a sufficient dosage level can give rise to the same IgG response.

1-11. (canceled)

12. A process for the manufacture of an immune system stimulant comprising the steps of:
(a) identifying and capturing a target pathogen;
(b) introducing target pathogens into a chamber;
(c) attenuating the pathogens by exposing said pathogens to hydroxyl radicals; and
(d) capturing the attenuated pathogens.

13. A process according to claim 12, including the further step of culturing the captured target pathogen to produce a significant number of targets.

14. A process according to claim 12, including the further step of sampling the attenuated pathogens following exposure to hydroxyl radicals.

15. A process according to claim 12, including the further step of testing the attenuated pathogens for active pathogens.

16. A process according to claim 12, including the further step of mixing the captured attenuated pathogens resulting from step (d) of claim 12 with a carrier fluid.

17. A process according to claim 12, including the further step of filling an administration device with the immune system stimulant.

18. An immune system stimulant for stimulating the production of antibodies against a pathogen comprising said pathogen attenuated by exposure to hydroxyl radicals.

19. An immune system stimulant according to claim 18, wherein the immune system stimulant is manufactured by the process of claim 12.

20. The use of a pathogen attenuated by exposure to hydroxyl radicals in one of a medicament or the manufacture of a medicament for the treatment of a condition caused by the said pathogen in its unattenuated form.

21. The stimulation of the immune system of an individual against attack by a pathogen by treating the individual with an immune stimulant according to claim 18.
